(19) United States
(12) Patent Application Publication
(10) Pub. No.: US 2023/0218284 A1
(43) Pub. Date: Jul. 13, 2023

(54) INFLATABLE BALLOON RETRACTOR WITH PRESSURE SENSING AND FEEDBACK CAPABILITIES FOR AVOIDANCE OF EXCESS APPLIED PRESSURE IN BRAIN SURGERY

(71) Applicant: Ryan PARK, Hinsdale, IL (US)

(72) Inventor: Ryan PARK, Hinsdale, IL (US)

(21) Appl. No.: 17/573,444

(22) Filed: Jan. 11, 2022

(51) Int. Cl. A61B 17/02; A61B 5/00; A61B 5/023; A61B 17/00

(52) U.S. Cl. CPC A61B 17/02; A61B 5/0002; A61B 5/023; A61B 2017/00238; A61B 2017/00557; A61B 2562/0252

(57) ABSTRACT

A pressure sensing balloon retractor for use in brain surgery to avoid mechanical injury to brain tissues. The balloon retractor includes an inflatable balloon that can be inserted in-between brain tissues to increase accessibility during surgery. A pressure transducer connected to a microcontroller senses the pressure of the retractor, and this retractor pressure is compared to the patient’s mean arterial pressure derived from a blood pressure monitor in order to determine whether the pressure exceeds the threshold for brain injury. A load cell can be used to calibrate the microcontroller to remove the effect of elastic pressure on the pressure transducer’s measurements.

### BACKGROUND

### 1. Technical Field

Embodiments of the present disclosure relate generally to a medical retractor, and more particularly to a balloon retractor with pressure-sensing capabilities.

### 2. Related Art

In a surgery, retractors are used to hold the edges of a surgical cut apart in order for the surgeon to access the area within the incision. In a brain surgery, for example, retractors come with a high risk of reduced blood flow (ischemia) or other mechanical injuries if excess pressure is applied by the retractor. Balloon retractors are a form of retractor where a balloon is inserted into the tissue and inflated to widen the space between tissues.

Therefore, retractors that allow an effective way of precisely controlling the pressure applied to the tissue are required to avoid injury in a surgery, especially in a brain surgery.

### BRIEF DESCRIPTION OF DRAWINGS

Features, aspects, and embodiments are described in conjunction with the attached drawings, in which:

FIG. 1 shows an overview of a balloon retractor according to an embodiment of the present invention;

FIG. 2A is an external view of a pressure control module according to an embodiment of the present invention;

FIG. 2B shows the internals of the same pressure control module featured in FIG. 2A according to an embodiment of the present invention;

FIG. 3A shows a fluid reservoir with a gear pump according to an embodiment of the present invention;

FIG. 3B shows the internals of the same gear pump featured in FIG. 3A according to an embodiment of the present invention; and,

FIG. 4 shows an example of how blood pressure data is sent to the microcontroller of the pressure control module featured in FIG. 2A according to an embodiment of the present invention;

FIG. 5 is a flowchart detailing the negative feedback loop that allows the balloon retractor to self-adjust, as well as the manual override system linked to the loop.

### DETAILED DESCRIPTION

Hereinafter, a pressure sensing balloon retractor according to an embodiment of the present invention will be described below with reference to the accompanying drawings.

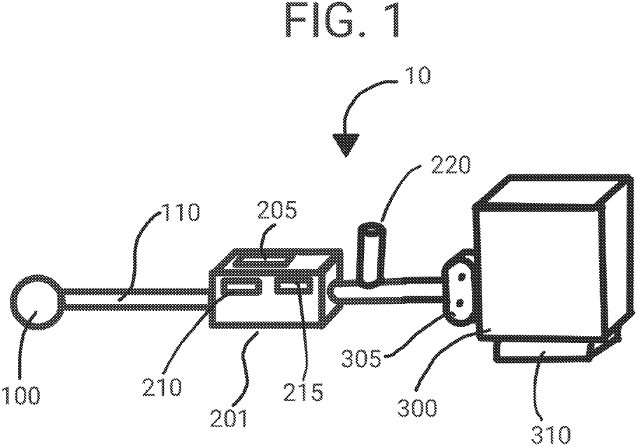

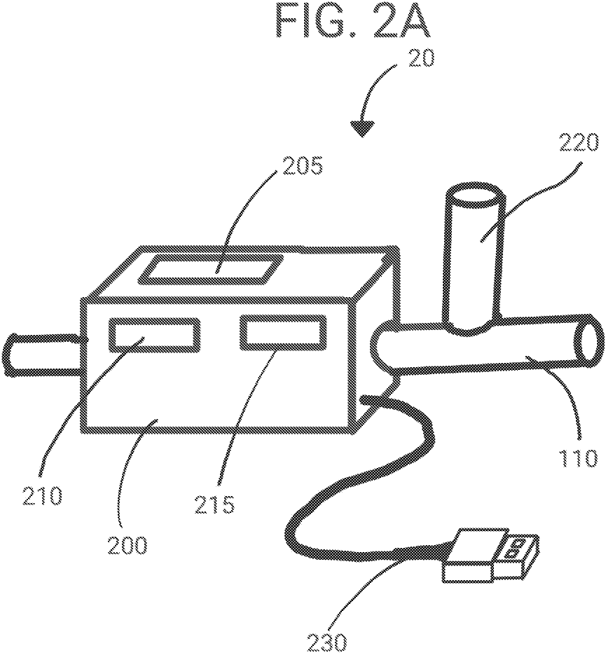

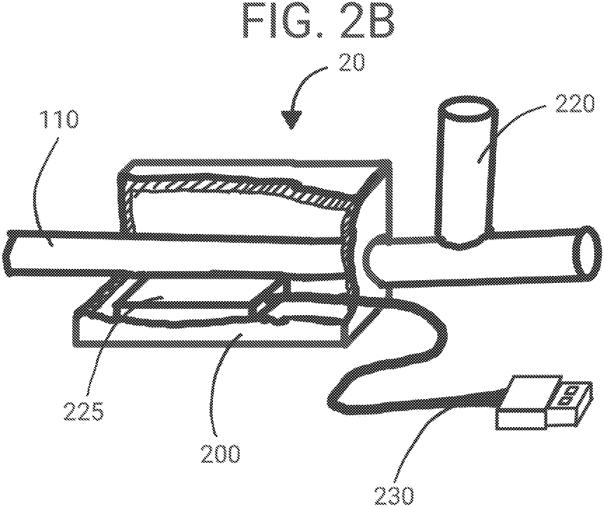

Referring to FIG. 1, the self-adjusting balloon retractor (10) includes, among others, a silicone balloon (100), a pressure control module (20, the label “20” is shown in FIG. 2A), and a fluid reservoir (300), all of which are connected by a flexible pipe (110).

Referring to FIG. 2A, the pressure control module (20) of FIG. 1 is shown in more detail. The pressure control module (20) is connected to the electronic pressure transducer (220). The pressure control module (20) includes a casing (200), which can be formed of plastic, metal, or other suitable material, along with an LCD electronic display screen (205), a “Fill” button (210), and an “Empty” button (215). Adjacent to the pressure control module (20) is an electronic pressure transducer (220) that detects the gage pressure within the fluid pipe (110) via detecting the change in electronic resistance caused by the bending of a pressure-sensitive membrane. The microcontroller is connected to an appropriate power source (230), which can be a direct electrical connection to an AC outlet, USB port, etc., or a connection to a battery.

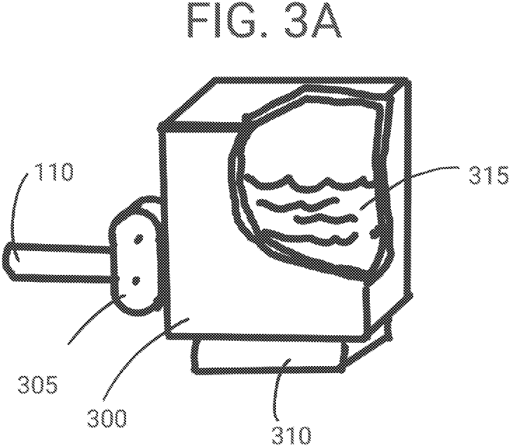

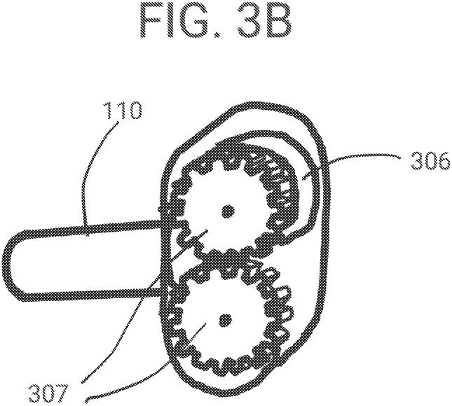

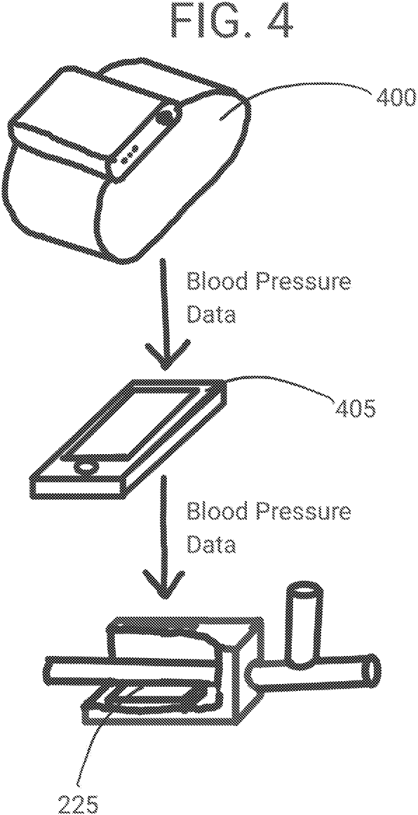

Referring to FIG. 2B, the pressure control module (20) featured in FIG. 2A contains a microcontroller (225) that is electronically connected to the LCD display (205), the pressure transducer (220), and the two or more buttons (210, 215) featured in FIG. 2A. The microcontroller (225) is also connected to the load cell (310) and pump (305) shown in FIG. 3A, and it also contains a Bluetooth module for wireless connection to a smartphone (400, featured in FIG. 4) or other suitable communication device either via wire or wirelessly.

Referring to FIG. 3A, the fluid reservoir (300) contains a fluid (315) which is pumped through the fluid pipe (110) via a gear pump (305). An electronic load cell (310) monitors the fluid weight of the reservoir.

Referring to FIG. 3B, the gear pump (305) featured in FIG. 3A contains two or more meshed gears (307) connected to an electronic motor (306). The spinning of the motors causes the gears to spin, and the motion of the gear teeth pushes fluid through the fluid pipe (110) in one of two directions. Even though a gear pump (305) is used in an embodiment of the present invention, other suitable fluid pump can be used instead. For example, referring to FIG. 3B, an alternative to the gear pump (305) would be an electric pump capable of moving fluid in two directions.

Referring to FIG. 4, the microcontroller (225) shown in FIG. 2B is wirelessly connected to a smartphone (405), which is wirelessly connected to a blood pressure monitor (400). Alternatively, the microcontroller (205) can be directly connected to a blood pressure monitor (400) by wire or wirelessly. The blood pressure monitor (400) detects the blood pressure of the patient undergoing surgery, sends the blood pressure information to the smartphone (405), and the smartphone (405) is able to send the data to the microcontroller (225) via the microcontroller’s Bluetooth module.

Referring to FIG. 4, an alternative to the wireless blood pressure (400) monitor is any blood pressure monitor capable of directly integrating with the microcontroller, rather than through wireless means.

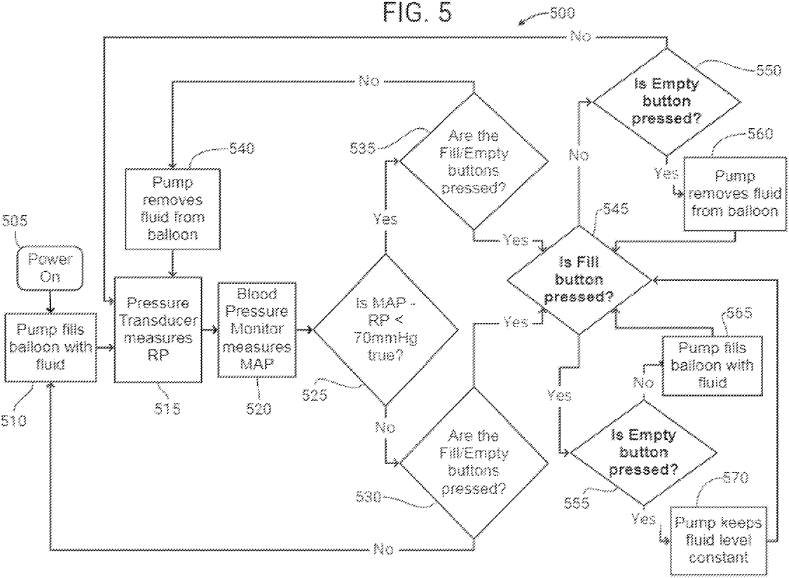

Referring to FIG. 5, when the system is activated (505), the microcontroller (225) is programmed to activate the gear pump (305) and send fluid from the reservoir to the silicone balloon (510). As the balloon fills up, the pressure transducer (220) is able to detect the changes in pressure (515) caused by the surrounding tissue pushing on the expanding balloon. When the pressure exceeds the minimum pressure threshold of brain injury (525), the microcontroller (225) adjusts the gear pump (305) to regulate the pressure in a negative feedback loop (535), ensuring that the retractor’s pressure does not exceed the threshold.

Referring to FIG. 5, the aforementioned minimum pressure threshold of brain injury is determined by measuring the mean arterial pressure (MAP) (520) of the patient and the pressure of the retractor itself (RP) (515). The RP is determined by the gage pressure measurement of the pressure transducer (220) in FIGS. 2A and 2B. The MAP is determined via the blood pressure readings derived from the blood pressure monitor in FIG. 4. The MAP can be calculated based on systolic (SYS) and diastolic (DIA) blood pressure measurements: MAP = (SYS + 2*DIA)/3. Therefore, by measuring the systolic and diastolic blood pressures of the patient undergoing brain surgery, that data can be transferred to the microcontroller via a smartphone as shown in FIG. 4, and the microcontroller can then calculate the corresponding MAP (520). The microcontroller can then calculate the difference between the MAP and RP (525). If this difference exceeds a certain threshold determined from research, then the microcontroller will control the gear pump to lower the pressure below the threshold, forming a negative feedback loop of pressure regulation that avoids brain injury. For example, the threshold of brain injury is when the difference between MAP and RP (MAP - RP) is less than 70 mmHg; therefore, in a brain surgery application, the microcontroller would be comparing the calculated difference between MAP and RP to the value of 70 mmHg to determine whether pressure in the balloon (100) should be increased or decreased (525). If the difference MAP - RP is less than 70 mmHg, then the RP must be too great, causing the microcontroller to decrease the RP (540) until the difference between MAP and RP is greater than 70 mmHg once again, avoiding injury to the tissues. If the difference MAP - RP is greater than 70 mmHg, then the RP is not dangerously high, causing the microcontroller to control the pump to continue to fill the balloon with more fluid (510).

Referring to FIG. 5, the operator of the device is able to manually override the microcontroller’s negative feedback (500) via the “Fill” (210) and “Empty” (215) buttons featured on the side of the pressure control module (20) in FIG. 2A. When either the “Fill” or “Empty” button is pressed (530, 535), the negative feedback loop is halted. If the “Fill” button is pressed alone (545), the microcontroller turns the gear pumps such that fluid from the reservoir fills and expands the balloon (565). When the “Empty” button is pressed alone (550), the microcontroller turns the gear pumps such that fluid exits the balloon and returns to the reservoir (560). The instantaneous retractor pressure (RP) and its difference from the calculated mean arterial pressure (MAP) are displayed on the LCD display screen (205) in order to inform the user about whether the retractor pressure is exceeding safe levels. If both buttons are pressed at once (545, 555), the amount of fluid in the balloon is kept constant (570). If neither of the buttons are being pressed (545, 550), then the negative feedback loop with automatic adjustment is restored. The manual override ensures that, in the case of a malfunction in the negative feedback loop, the user can manually override the loop to ensure that no brain injury occurs.

The load cell (310) is used to account for the pressure of the balloon (100) itself when measuring retractor pressure (RP). When the balloon is inserted into brain tissue, the pressure measured by the pressure transducer (220) is due to the sum of the elastic pressure of the balloon (100) and the pressure of the surrounding brain tissue on the retractor. In order to remove the effect of the balloon’s elastic pressure, the retractor can be calibrated before use. Calibration is performed by gradually filling the balloon (100) with fluid outside of tissue, recording the pressures from the transducer (220), and using the load cell (310) to monitor the amount of mass of fluid that was inserted into the balloon. This allows the microcontroller (225) to obtain a control data set relating the amount of mass of fluid in the balloon to the pressure of the balloon outside of tissue, which represents the pressure due to the balloon’s elasticity alone. When the retractor balloon is inserted into brain tissue after calibration, the resultant pressure data can then be subtracted from the control data set (from calibration) for a given mass of fluid (from the load cell, 310, in FIG. 3A) in order to remove the effects of elastic pressure, more accurately measuring the pressure of the balloon retractor itself on the brain tissue.

This device could be used as an adjustable catheter. For example, the device could be used as a urinary catheter for the purpose of draining urine from a patient’s bladder via insertion and inflation of the retractor balloon (100) in the bladder.

Although specific advantages have been enumerated above, various embodiments may include some, none, or all of the enumerated advantages.

Other technical advantages may become readily apparent to one of ordinary skill in the art after review of the following figures and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise specifically noted, articles depicted in the drawings are not necessarily drawn to scale.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

While certain embodiments have been described above, it will be understood to those skilled in the art that the embodiments described are by way of example only. Accordingly, the pressure sensing balloon retractor described herein should not be limited based on the described embodiments. Rather, the pressure sensing balloon retractor described herein should only be limited in light of the claims that follow when taken in conjunction with the above description and accompanying drawings.

What is claimed is:

1. A self-adjusting balloon retractor to adjust and maintain an incision gap of a patient in a surgery, the self-adjusting balloon retractor comprising:
an inflatable balloon configured to adjust a space between tissues to create desired space suitable for performing the surgery, wherein the balloon is inflated by controlling the amount of fluid therein; and
a microcontroller controlling the fluid flow to and from the balloon based on an internal fluid pressure data of the balloon and a blood pressure of the patient, wherein a negative feedback loop is maintained such that the microcontroller receives pressure data from a pressure transducer, compares the data to an estimated arterial pressure based on patient blood pressure data, and adjusts a fluid pump accordingly to increase or decrease the retractor’s internal fluid pressure such that injury to surrounding tissues due to excess pressure is avoided;
a pressure transducer that senses the internal fluid pressure within the apparatus and sends the pressure data to the microcontroller;
a pump connected to a container of fluid and a pipe such that the microcontroller can control the flow of fluid through the pipe via controlling the pump;
a fluid reservoir connected to the balloon via a pipe;
an electronic load cell located under the fluid reservoir and electrically connected to the microcontroller, wherein the load cell sends fluid weight data to the microcontroller;
a wireless blood pressure monitor connected to the microcontroller wirelessly, either directly or via a smartphone as an intermediate, wherein the monitor sends blood pressure data to the microcontroller;
a plastic casing comprising:
a plurality of buttons, each of which has the purpose of manually controlling the pump;
an electronic display that displays information relevant to the operation of the device.

2. The self-adjusting balloon retractor of claim 1, wherein the retractor comprises a retractor for use in brain surgery.

3. The self-adjusting balloon retractor claim 1, wherein the pump comprises an external gear pump containing two counterrotating gears.

4. The self-adjusting balloon retractor of claim 1, wherein the transducer comprises a piezoelectric pressure transducer.

5. The self-adjusting balloon retractor claim 1, wherein the balloon comprises a silicone bulb.

6. The self-adjusting balloon retractor of claim 1, wherein the monitor comprises a commercially available wireless blood pressure device capable of Bluetooth connection.

7. The self-adjusting balloon retractor claim 1, wherein the power supply of the microcontroller further comprises:
a connection to a battery;
a connection to an external power source;
.

8. The self-adjusting balloon retractor of claim 1, wherein the negative feedback loop further comprises:
a mathematical formula MAP = (SYS + 2*DIA)/3 used to derive the MAP from obtained systolic (SYS) and diastolic (DIA) blood pressure measurements from a patient;
a mathematical formula MAP - RP < 70 mmHg used to determine the point when the pressure of the retractor (RP) reaches dangerously high levels;
a control mechanism to manually override the negative feedback loop, wherein pressing a plurality of buttons enables a user to manually control the pressure of the retractor.

9. A method of operating a self-adjusting balloon retractor to adjust and maintain an incision gap of a patient in a surgery, the method comprising:
adjusting a size of an inflatable balloon by controlling the amount of fluid, wherein the inflatable balloon is configured to create a space between tissues suitable for performing the surgery; and
controlling fluid flow to and from the balloon using a microcontroller based on an internal fluid pressure data of the balloon and a blood pressure of the patient,
wherein a negative feedback loop is maintained such that the microcontroller receives pressure data from a pressure transducer, compares the data to an estimated arterial pressure based on patient blood pressure data, and adjusts a fluid pump accordingly to increase or decrease the retractor’s internal fluid pressure such that injury to surrounding tissues due to excess pressure is avoided;
sensing the internal fluid pressure within the apparatus and sending the pressure data to the microcontroller by using a pressure transducer;
controlling the fluid flow through the pipe via controlling the pump by using the microcontroller,
wherein a pump is connected to a container of fluid and a pipe, and
wherein a fluid reservoir is connected to the balloon via a pipe, and
wherein an electronic load cell is located under the fluid reservoir and electrically connected to the microcontroller, such that the load cell sends fluid weight data to the microcontroller, and
wherein a wireless blood pressure monitor is connected to the microcontroller wirelessly, either directly or via a smartphone as an intermediate, wherein the monitor sends blood pressure data to the microcontroller;
wherein a plastic casing comprises:
a plurality of buttons, each of which has the purpose of manually controlling the pump;
an electronic display that displays information relevant to the operation of the device.
